SEQUENCE LISTING (1 sequence)

SEQ ID NO: 1 - protein, 11 aa, Homo sapiens
RPKPQQFFGLM

(19) United States
(12) Patent Application Publication
(10) Pub. No.: US 2007/0155667 A1
(43) Pub. Date: Jul. 5, 2007

(54) Acute respiratory syndromes

(75) Inventor: Mark L. Witten, Tucson, AZ (US)

Correspondence Address: BANNER & WITCOFF, LTD., 1100 13th STREET, N.W., SUITE 1200, WASHINGTON, DC, 20005-4051, US

(73) Assignee: IMMUNEREGEN BIOSCIENCES, INC., SCOTTSDALE, AZ (US)

(21) Appl. No.: 10/553,232

(22) PCT Filed: Apr. 14, 2004
(86) PCT No.: PCT/US04/11399

§ 371 Date: Oct. 3, 2006

(30) Foreign Application Priority Data
Apr. 14, 2003 (US) 60462316
Apr. 25, 2003 (US) 60465266

(51) Int. Cl. A61K 38/08

(52) U.S. Cl. 514/15

(57) ABSTRACT

Substance P or its analogs are useful for treating and protecting against acute respiratory syndromes including ARDS and SARS. The active agents can be administered via inhalation therapy, intravenously, intramuscularly, sublingually, or by other methods. Disease indicia are reduced by substance P treatment.

This application claims the benefit of provisional application Ser. No. 60/462,316 filed Apr. 14, 2003 and provisional application Ser. No. 60/465,266 filed Apr. 25, 2003.

### BACKGROUND OF THE INVENTION

Acute Respiratory Distress Syndrome (ARDS) is characterized by a rapid influx of body fluids from the capillaries to the lung alveolar spaces. ARDS can be caused by acute smoke inhalation, respiratory viral illness, acute trauma, and aspiration of stomach of stomach contents.

Severe Acute Respiratory Syndrome (SARS) is characterized by fever, chills, myalgia, and cough. Respiratory symptoms and auscultatory findings are fairly mild, compared to radiographic changes observed of the chest. A virus belonging to the family Coronaviridae was isolated from two SARS patients. By use of serological and reverse-transcriptase PCR specific for this virus, patients with SARS, but no controls, had evidence of infection with this virus. Other corona viruses are involved in causing the common cold. High concentrations of corona viral RNA (≦100 million molecules per milliliter (10⁸/ml) have been found in sputum of infected patients. Viral RNA was also detected at extremely low concentrations in plasma during the acute phase and in feces during the late convalescent phase.

### SUMMARY OF THE INVENTION

According to one embodiment of the invention a method is provided for treating a SARS or ARDS patient. An effective amount of an agent selected from the group consisting of: substance P, [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met (0₂)11-Substance P, and [p-Cl-Phe^{7,8}]-substance P is administered to the patient. A disease feature selected from the group consisting of: Clara cell necrosis, pulmonary alveolar macrophage number, alveolar-capillary barrier membrane damage, and 6-keto-PGF_{1α} and PGE₂ concentration is thereby decreased.

According to another aspect of the invention a method is provided for protecting an individual from developing SARS or ARDS. The individual has been or is expected to be exposed to a patient with SARS or ARDS. An effective amount of an agent selected from the group consisting of: substance P. [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met (0₂) 11-Substance P, and [p-Cl-Phe^{7,8}]-substance P is administered to the individual. The risk of developing SARS or ARDS is thereby reduced.

### DETAILED DESCRIPTION OF THE INVENTION

It is a discovery of the present inventor that Substance P and its bioactive analogs, such as Sar⁹, Met (0₂) 11-Substance P, is a beneficial treatment for ARDS, corona virus respiratory infections, and corona-like respiratory virus infections. Substance P and its analogs also potentiate the lung immune system's response against corona and corona-like respiratory viruses. Substance P and its analogs can be used to prophylactically treat health care workers and family members who must care for SARS patients and suspected SARS patients.

Substance P (RPKPQQFFGLM-NH₂; SEQ ID NO: 1) or a bioactive analog thereof such as Sar⁹, Met (0₂) 11-Substance P can be administered to treat ARDS, SARS, corona and corona—like respiratory virus infections. The bioactive analog can be selected from the group consisting of [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met (0₂) 11-Substance P, and [p-Cl-Phe^{7,8}]-substance P. Other compounds which function in the same way can be identified by their ability to compete with substance P for binding to its receptor (NK-1) or for their ability to agonize the NK-1 receptor. Routine assays for such activities are known in the art and can be used.

The substance P or analog can be administered by any method known in the art, including via aerosol inhalation. Intravenous, intratracheal, intrabronchial, intramuscular, sublingual, and oral administrations can also be used. Preferred dosages include 0.05 to 5 nanomolar substance P or analog for intravenous administration, preferably 0.1 to 2 nanomolar, and more preferably 0.5 to 1.5 nanomolar. For aerosol administration dosages include 0.05 to micromolar substance P or analog, preferably 0.1 to 2 micromolar, and more preferably 0.5 to 1.5 micromolar. Typical concentration ranges of substance P or its bioactive analog in the aerosol administered is between 0.001 and 10 μM. It can be advantageously administered as a liquid at a concentration between about 0.1 and 10 μM.

Bioactive analogs, according to the invention are those which act as competitive inhibitors of SP by binding to the SP receptor (NK-1 receptor). The analogs may be agonists of the NK-1 receptor. Other derivatives as are known in the art and commercially available (e.g., from Sigma) can be used. In addition, substance P fragments and derivatized substance P fragments may also be used. Substitution, deletion, or insertion of one to eight amino acid residues, and preferably from one to three amino acid residues, will lead to analogs which can be routinely tested for biological activity. In addition, functional groups may be modified on SP while retaining the same amino acid backbone. Again, routine testing will determine which of such modifications do not adversely affect biological activity.

Suitable devices for administering the aerosol of the present invention include nebulizers as well as hand-held aerosol “puffer” devices. Suitable treatment regimens for treatment according to the present invention include daily or multiple daily treatment by aerosol. Other modes of treatment include continual transdermal infusion, intravenous injection, intramuscular, sublingual, subcutaneous injection, and oral administration. Suitable formulations of substance P for administration are any which are pharmaceutically acceptable and in which substance P retains its biological activity. Generally, such formulations are substance P dissolved in normal sterile saline. Other formulations for changing absorption and half-life characteristics can be used, including liposomal formulations and slow-release formulations.

Disease features of ARDS and SARS include Clara cell necrosis, increased pulmonary alveolar macrophage number, neutrophil number, alveolar-capillary barrier membrane damage, and increased 6-keto-PGF_{1α} and PGE₂ concentrations. These disease features are reduced by the therapeutic administrations of the present invention. Decreases in the disease features of at least 10%, 15%, 20%, 25%, 30%, 35%, 40%, or 50% are desirable. Even greater decreases are preferred.

### EXAMPLE 1

We have developed a model of ARDS and SARS in rats and rabbits. We demonstrated that 30 tidal volume breaths of diesel fuel smoke caused a persistent increase in lung substance P levels of up to 60% for 24 hours post-smoke. The lung injury was also characterized by clara cell necrosis, increase in pulmonary alveolar macrophages, and increases in the prostaglandins; 6-keto-PGF_{1α} and PGE₂. Treatment with intravenous (0.8 nM) Sar⁹, Met (0₂) 11-Substance P attenuated the clara cell necrosis, reduced pulmonary alveolar macrophage number, and decreased 6-keto-PGF_{1α} and PGE₂. Such treatment attenuates damage to the alveolar-capillary barrier membrane. Thus Sar⁹, Met (0₂) 11-Substance P is a beneficial treatment for ARDS, corona, and corona-like respiratory viruses.

I claim:

1. A method of treating a SARS or ARDS patient, comprising:
administering to the patient an effective amount of an agent selected from the group consisting of: substance P, [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met (0₂) 11-Substance P, and [p-Cl-Phe^{7,8}]-substance P, whereby a disease feature selected from the group consisting of: Clara cell necrosis, pulmonary alveolar macrophage number, neutrophil number, alveolar-capillary barrier membrane damage, and 6-keto-PGF_{1α} and PGE₂ concentration is decreased.

2. The method of claim 1 wherein the patient is a SARS patient.

3. The method of claim 1 wherein the patient is an ARDS patient.

4. The method of claim 1 wherein Sar⁹, Met (0₂) 11-Substance P is administered.

5. The method of claim 1 wherein Substance P is administered.

6. The method of claim 1 wherein the step of administering is performed by inhalation of an aerosol.

7. The method of claim 1 wherein the step of administering is performed by intravenous delivery.

8. The method of claim 1 wherein the step of administering is performed by intramuscular delivery.

9. The method of claim 1 wherein the step of administering is performed by sublingual delivery.

10. A method of protecting an individual prior to or after exposure to a patient with SARS or ARDS from developing SARS or ARDS, comprising:
administering to the individual an effective amount of an agent selected from the group consisting of: substance P, [Met-OH¹¹]-substance P, [Met-OMe¹¹]-substance P, [Nle¹¹]-substance P, [Pro⁹]-substance P, [Sar⁹]-substance P, [Try⁸]-substance P, Sar⁹, Met (0₂) 11-Substance P, and [p-Cl-Phe^{7,8}]-substance P, whereby risk of developing SARS or ARDS is reduced.

11. The method of claim 10 wherein the agent is substance P.

12. The method of claim 10 wherein the agent is Sar⁹, Met (0²) 11-Substance P.

13. The method of claim 10 wherein the step of administering is performed by inhalation of an aerosol.

14. The method of claim 10 wherein the step of administering is performed by intravenous delivery.

15. The method of claim 10 wherein the step of administering is performed by intramuscular delivery.

16. The method of claim 10 wherein the step of administering is performed by sublingual delivery.
